(19) United States
(12) Patent Application Publication
(10) Pub. No.: US 2017/0354750 A1
(43) Pub. Date: Dec. 14, 2017

(54) TREATMENT CHAMBER MADE IN LIGHT-WEIGHT CONSTRUCTION

(71) Applicant: B. BRAUN MELSUNGEN AG, MELSUNGEN (DE)

(72) Inventors: RALPH GEIGER, FELSBERG (DE); CHRISTIAN GUNDLACH, FULDABRÜCK (DE)

(21) Appl. No.: 15/606,702

(22) Filed: May 26, 2017

(30) Foreign Application Priority Data
Jun. 8, 2016 (DE) 102016110572.4

(51) Int. Cl. A61L 2/00; F25D 11/00; A61G 13/10; A61L 2/26; A61B 50/36; A61B 50/00; A61B 50/30

(52) U.S. Cl. CPC A61L 2/0023; A61L 2/26; F25D 11/003; A61B 50/36; A61G 13/108; A61B 2050/314; A61B 2050/001

(57) ABSTRACT

A treatment chamber, preferably a medical treatment chamber for treating articles, preferably medical articles, devices and equipment, more particularly within the scope of sterilizing processes, including a treatment compartment which is surrounded by a lower wall element, a plurality of lateral wall elements and an upper wall element, wherein the treatment compartment is hermetically sealed or sealable for generating a desired internal pressure therein and can be temperature-controlled for generating a desired internal temperature therein, wherein at least one of the wall elements is formed of at least one hollow chamber section is disclosed.

### CROSS REFERENCE TO RELATED APPLICATIONS

This application claims priority to German application DE 10 2016 110 572.4 filed Jun. 8, 2016, the contents of such application being incorporated by reference herein.

### FIELD OF THE INVENTION

The present invention relates to a treatment chamber, preferably a medical treatment chamber, for treating articles, preferably medical articles, instruments, devices and other equipment, preferably for treating medical single-use articles, more particularly within the scope of sterilizing processes, comprising a treatment volume or treatment compartment which is surrounded by a lower wall element or a bottom, a plurality of lateral wall elements and an upper wall element or a ceiling, wherein the treatment compartment is hermetically sealed or sealable for generating a desired internal pressure therein and can be temperature-controlled for generating a desired internal temperature therein.

### BACKGROUND OF THE INVENTION

During manufacture of articles, for example medical sterile articles, especially of medical sterile single-use articles, different chambers are used for treating the articles in terms of the production process, inter alia sterilization chambers for sterilizing the articles or drying chambers for drying the articles, in particular by utilizing a combination of heat and vacuum. Usually said chambers are welded from solid material plates of stainless steel and are heated by a fluid such as water or air via a cover. By the use of vacuum in the sterilization process the chambers are exposed to high stresses and therefore are structurally supported and provided with reinforcements. This renders the entire constructional design and manufacture of such chambers very complicated and requires high material input, as plates or sheet metals of sufficient thickness as well as self-supporting elements for reinforcement, support and securing are required. The production cost and the material input are very high. Also, the delivery times are usually long. Delivery times of up to nine to twelve months are quite common.

During operation of a sterilization chamber according to prior art furthermore large quantities of energy are necessary to heat the chamber to a sufficient processing temperature. The solid design including structural reinforcing and supporting elements renders the mass to be temperature-controlled big. Moreover, the weight of the chamber usually is detrimentally very high (a chamber having a capacity for 20 pallets weighs approx. 20 t), which constitutes a high floor load for a building in which the chamber is provided and is related with high requirements to the statics of the building.

In total a drawback of known chambers is constituted by high material input, high energy expenditure, high weight, high energy consumption, high expenditure of manual work as well as long delivery times.

### SUMMARY OF THE INVENTION

On this basis, an object underlying aspects of the present invention is to provide a chamber for producing medical products, especially single-use medical products, which does not have said drawbacks. The chamber is especially intended to be light-weight and to require a small amount of energy, especially less energy than comparable known systems. It is desirable when the chamber can be produced to be flexibly adapted as to its dimensions to circumstances present at the respective production site at low cost. Furthermore, it is intended to be simply manufactured by common production means.

This object is achieved, according to aspects of the present invention, by a treatment chamber or processing chamber, preferably a medical treatment chamber, especially a sterilization chamber or desorption chamber, in accordance with the preamble of the independent claim, wherein at least one of the wall elements is made of at least one hollow structure section or hollow chamber section.

Within the scope of the present description of the invention, a wall element is meant to be any component delimiting the treatment volume or the treatment compartment, irrespective of its respective orientation or positioning. Such wall element may form, apart from sidewalls, furthermore a bottom, a ceiling, a cover or a door or any other closing means.

The use according to aspects of the invention of a hollow chamber section allows the treatment or processing chamber to be manufactured in an especially advantageous manner in light-weight construction. The saving of material in the hollow chamber sections renders processing chambers according to aspects of the invention definitely more light-weight with respect to conventional processing chambers while having the same capacity due to the low weight of the hollow chamber sections so that static requirements to buildings in which the chambers are set up are lower. On the other hand, due to their internal structure and the trussed-type design hollow chamber sections offer excellent static and dynamic properties such as e.g. bending resistance, torsional stability or load-bearing capacity.

The hollow chamber sections employed within the scope of the invention advantageously have a simple design, low weight and can be easily and inexpensively manufactured e.g. as extruded sections of numerous different sectional geometries. The required material input for constructing such hollow chamber section and thus a wall element formed of the same is low. Especially the mass of the section and of the wall element is low, which is advantageous not only in terms of weight. A wall element consisting of hollow chamber sections and thus a processing chamber consisting of said wall elements require relatively low energy flows for heating and cooling due to the low mass, and therefore enable more rapid heating and cooling and a more uniform heat distribution than this is the case with known processing chambers.

It is a particular advantage that the wall elements as well as the processing chamber consisting thereof can be conceived and implemented in modular design. Plural hollow chamber sections may be combined and composed to form a wall element. Depending on the number of hollow chamber sections used for this purpose, wall elements of different dimensions can be easily manufactured. By using hollow chamber sections of appropriate length, the length of the wall elements composed thereof can be determined. The modular design of the chamber according to aspects of the invention enables quick assembly, use of pre-fabricated standard parts, short manufacturing time resulting in short delivery time, easy transportation and low energy input.

Despite their low weight, hollow chamber sections have high stability which can be specifically determined in terms of constructions by the geometry of the section, especially by its geometry in cross-section, and can be specifically designed. In particular, in the transverse direction the hollow chamber section may absorb high loads without deforming in an undesired way. A wall element consisting of hollow chamber sections thus meets the pressure requirements existing within the scope of sterilization processes, desorption processes and especially low-pressure or vacuum processes.

Advantageous embodiments of the invention are claimed in the dependent claims and shall be explained in detail hereinafter.

One embodiment of the invention is wherein the hollow chamber section is an extruded section. Especially advantageously, it is an extruded aluminum section. The use of aluminum as material is possible by the lower material input and the high structural strength. Aluminum, as material, offers significant physical advantages with respect to stainless steel which has been used for such processing chambers so far. In this context, inter alia high thermal capacity, good thermal conductivity as well as low weight have to be mentioned.

Another embodiment of the invention is wherein the hollow chamber section comprises an internal structure of cross braces or an internal honeycomb structure. The cross braces or walls of the honeycombs configure sort of a support structure which while having a low weight ensures high stability and rigidity of the section. Between the braces and/or walls in the hollow chamber section continuous passages may be formed which are fluid-communicated so as to form a line system for a temperature-controlling fluid, a treatment fluid or an air-conditioning fluid. Hence it can be said that the honeycomb structure can be additionally utilized as heating passage or temperature-controlling passage for the walls, which is not the case in known chambers made from stainless steel comprising solid material walls.

According to one embodiment of the invention, in the hollow chamber section a continuous passage or plural continuous passages may be formed. The latter can be fluid-communicated with passages of adjacent hollow sections or other passages of the hollow section to form a line system for a temperature-controlling fluid, a treatment fluid or an air-conditioning fluid. In this context, the term continuous means that the passage forms a flow path through the hollow chamber section from an inlet to an outlet.

According to aspects the invention, plural hollow chamber sections may be connected or connectable by form closure and/or force closure. It is especially advantageous when the hollow chamber section exhibits a connecting structure, especially in the way of a tongue-and-groove system. The hollow section can be connected by the latter to other components or to a neighboring hollow section of the same wall element or of an adjacent wall element in a fluid-tight manner. The connecting structure is preferably formed at an end face of the hollow section. Preferably, on the respective opposite end face of the hollow section a counterpart matching the connecting structure is formed. The connecting structure and its counterpart may include an engaging structure which ensures stable coherence of sections joined by the same, at least as long as they have not yet been welded to each other or otherwise connected in the long run.

Corner connections of adjacent wall elements can be brought about, according to aspects of the invention, by the fact that on the border side of a wall element a hollow chamber section in the form of a corner section is arranged. The latter has a bend or an angle, preferably a 90° bend so that the opposing connecting structures are equally offset against each other by an angle of 90°.

Especially, the connecting structure may form a sliding seat to compensate for tolerances for a welding assembly. Said sliding seat may be configured, according to aspects of the invention, e.g. by the fact that interacting connecting structures as well as the optionally present engaging structures thereof are provided with inlet bevels and outlet bevels and/or have a particular resilience and thus are elastically deformable within certain limits without the engaged connecting structures disengaging from each other. It can also be said that the connecting structures enable a particular play of hollow chamber sections joined by the former due to their elasticity.

Plural hollow chamber sections in the horizontal and, respectively, vertical orientation can be connected to each other by material adhesion according to aspects of the invention. Such adhesive connection may be established alternatively or additionally to a form-fit and/or force-fit connection. In order to obtain a stable and especially fluid-tight processing chamber the hollow structure sections are preferably welded to each other. Further adhesive connections are possible and are within the scope of the invention, such as gluing or soldering. Furthermore, hollow chamber sections can be screwed to each other. For further simplified assembly, pre-fabricated components of plural already interconnected hollow chamber sections can be connected to form wall elements.

In particular, the following advantages can be achieved by the invention: due to the modular structure, different chamber sizes can be quickly manufactured. Sections of different heights and widths are easy to manufacture and to combine. The sections can be easily cut to length. The number and the length of the individual profiles result in different chamber sizes. In total, a modular design is facilitated.

Summing up, it can be stated that the invention enables processing chambers, especially sterilization and desorption chambers, which have low inherent weight due to the use of a honeycomb structure. A modular design renders the manufacture and the structure of a chamber according to aspects of the invention definitely simpler and thus more inexpensive. Structural requirements based on pressure differences relating to the process can be met by the honeycomb structure better than in prior art chambers. The invention especially permits to construct a sterilization or desorption chamber with the aid of extruded aluminum sections. A honeycomb structure of the sections helps to achieve maximum stability with minimum possible weight. Moreover, cavities of the structure or sections can be used for heating or cooling or air-conditioning the chamber. Taking the statics into consideration, chambers of different sizes can be quickly realized.

### BRIEF DESCRIPTION OF THE DRAWINGS

The invention is best understood from the following detailed description when read in connection with the accompanying drawings. Included in the drawings are the following figures:

FIG. 1 shows a perspective representation of a processing chamber according to aspects of the invention without an end-side wall element;

FIG. 2 shows a sectional view of the processing chamber of FIG. 1 along the line II-II;

FIG. 3 shows an enlarged view of a corner area of the sectional view of FIG. 2;

FIG. 4 shows an enlarged view of the connecting area of three neighboring hollow chamber sections; and

FIG. 5 shows a perspective representation of pre-fabricated interconnected components.

### DETAILED DESCRIPTION OF THE PREFERRED EMBODIMENTS

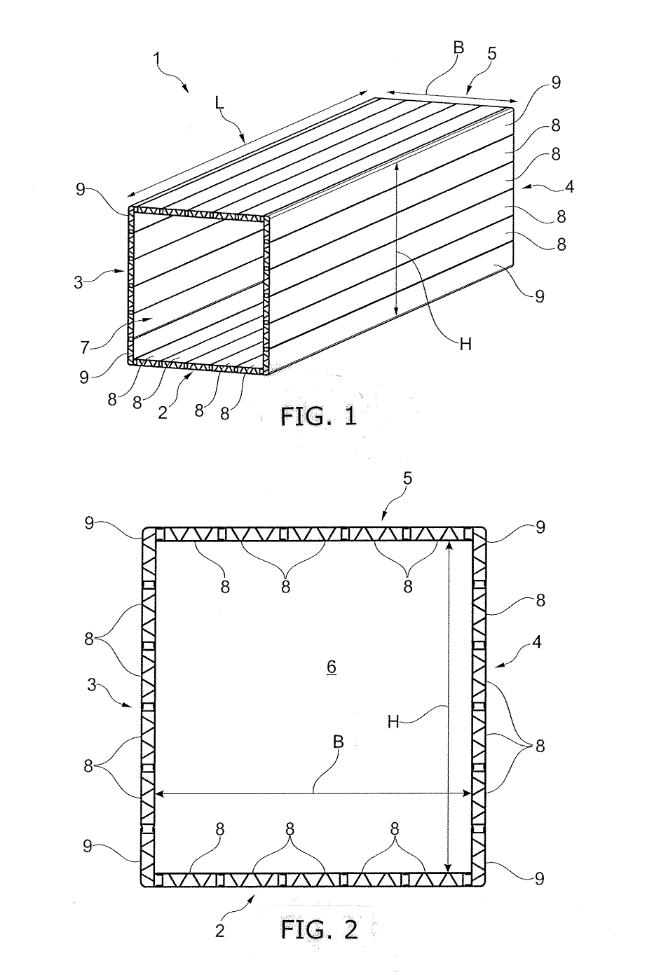

FIG. 1 shows a processing chamber 1 according to aspects of the invention in a perspective schematic view. The chamber 1 shows, as illustrated and visible in FIG. 1, a bottom wall 2, two side walls 3, 4 and a ceiling wall 5. A rear end wall 6 in FIG. 1 is not evident due to the perspective representation in FIG. 1, but it is indicated in FIG. 2. A front end wall or door which closes the end opening 7 located between the wall elements 2, 3, 4, 5 is not shown for reasons of clarity. Each of the afore-mentioned six walls forms a wall element in accordance with the invention.

According to aspects of the invention, each of the wall elements 2, 3, 4, 5 is composed, as a light-weight component, of a plurality of hollow chamber sections 8, 9. Each of the hollow chamber sections 8, 9 has an inner wall 10 and an outer wall 11 opposite thereto. Between the inner wall 10 and the outer wall 11 cross braces or sectional braces 12 are arranged. Since, in the present embodiment, the hollow chamber section 8, 9 is an extruded aluminum section, each of the sectional braces 12 is formed to be adhesively connected both to the inner wall 10 and to the outer wall 11. The sectional braces 12 extend continuously in the longitudinal direction L of the hollow chamber section 8, 9 and are arranged in the manner of a zigzag pattern (viewed in cross-section).

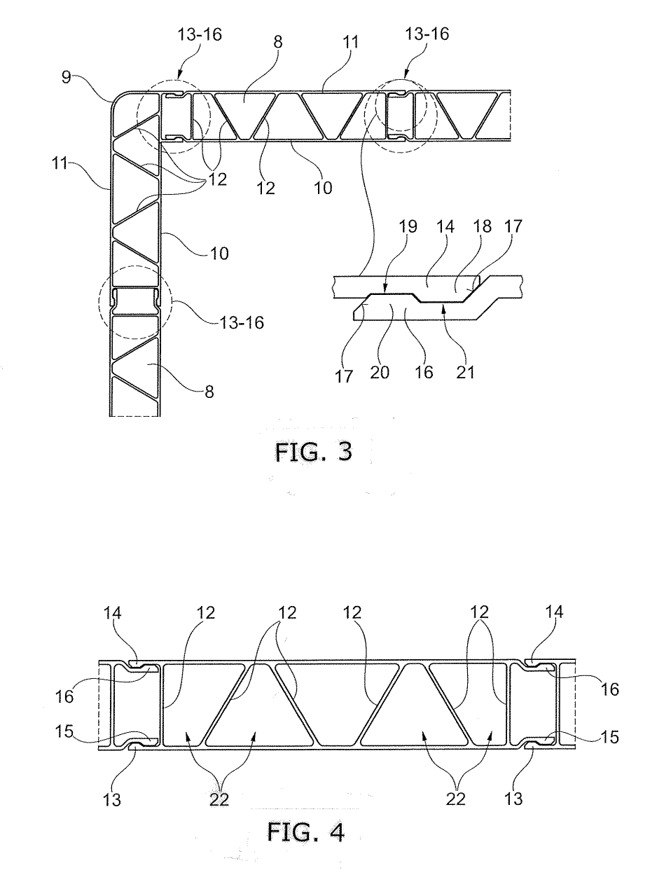

Especially from FIG. 3 it is clearly evident that the hollow chamber sections 8, 9, on the one hand, are wall hollow chamber sections 8 and, on the other hand, corner hollow chamber sections 9.

The wall hollow chamber sections 8 include connecting structures 13, 14, 15, 16 at the end face between the end portions of the inner wall 19 and the outer wall 11. On the one hand, (see FIG. 4) an inner groove element 13 and an outer groove element 14 are formed. On the other hand, i.e. on the opposite side, an inner tongue element 15 and an outer tongue element 16 are formed. All of said connecting structures at the inlet side have an inlet bevel 17 intended to facilitate joining of adjacent hollow chamber elements 8, 9. Each of the inner groove element 13 and the outer groove element 14 has at the end side an inwardly facing groove engaging head 18 as well as a connected groove engaging recess 19. Each of the inner tongue element 15 and the outer tongue element 16 at the end side includes an outwardly facing tongue engaging head 20 as well as a connected tongue engaging recess 21. As is shown in FIG. 4, the groove engaging head 18 engages in the tongue engaging recess 21 by form closure, while the tongue engaging head 20 engages in the groove engaging recess 19 by form closure. The corner hollow chamber sections 9 have a similar structure, with the difference that the groove elements 13, 14 are not arranged at the end side but on the inner wall 10. As a matter of course, instead of the groove elements 13, 14 the tongue elements 15, 16 can be arranged on the inner wall 10.

Each of the side faces of the groove engaging recess 19 as well as of the tongue engaging recess 21 (and thus also the side faces of the groove engaging head 18 and the tongue engaging head 20) is inclined, in the present example about an angle of 45°. All connecting structures 13, 14, 15, 16 are resilient arms which, due to their elasticity, are resilient either inwardly (tongue engaging elements) or outwardly (groove engaging elements). Due to their elasticity and the inclined side faces, in joined hollow chamber sections 8, 9 a kind of sliding seat is formed by the connecting structures 13, 14, 15, 16 for compensating for tolerances during weld mounting.

Between the cross braces 12 of the inner wall 10 and the outer wall 11 cavities 22 (or honeycombs or passages) are formed. They may be formed in total or partly as passages for a temperature-controlling fluid, a treating fluid or an air-conditioning fluid and may constitute a line system for the former by which the respective fluid is guided through the wall elements 2, 3, 4, 5, 6. The latter can be temperature-controlled in a simple and efficient manner, especially heated and/or cooled, so that the interior of the processing chamber 1 can be temperature-controlled or air-conditioned or—in the case of several passages being fluid-communicated with the interior—a particular protective gas can be applied to the same.

As is especially clearly evident from FIG. 1, the length L of the chamber 1 can be defined by appropriately cutting the hollow chamber sections to length. As is evident from FIG. 2, the height H and the width B of the chamber 1 can be varied by varying the number of the hollow chamber sections 8, 9 used.

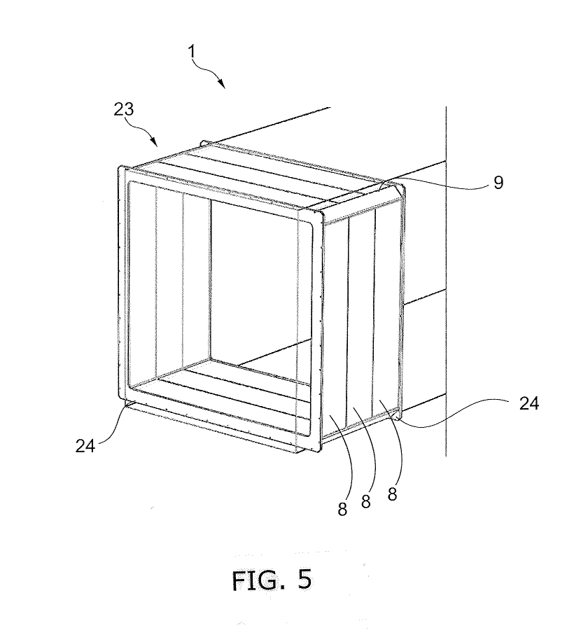

For the purpose of simplified assembly, plural hollow chamber sections 8, 9 can be connected ex works already to form pre-fabricated wall elements 23 so as to reduce the number of elements provided for local assembly. FIG. 5 illustrates a perspective view of such interconnected pre-fabricated components.

In the illustrated example, plural hollow chamber sections 8, 9 have been connected to form a rectangular hollow section. Plural pre-fabricated wall elements 23 designed as rectangular hollow sections then may be arranged one behind the other and may be connected to form the chamber 1. Instead of rectangular hollow sections, also flat pre-fabricated wall elements 23 or any imaginable combination of flat wall elements including corner hollow chamber sections 9 (e.g. U-sections or L-sections) are imaginable as pre-fabricated wall elements 23. For connecting the pre-fabricated wall elements 23 or for arranging a front end wall or door, flanges 24 may be provided at the end faces of the pre-fabricated wall elements.

1.-10. (canceled)

11. A treatment chamber for treating articles, the treatment chamber comprising:
a treatment compartment surrounded by a lower wall element, a plurality of lateral wall elements and an upper wall element, wherein the treatment compartment is hermetically sealed or sealable for generating a desired internal pressure therein and is temperature-controlled for generating a desired internal temperature,
wherein at least one of the wall elements is formed of at least one hollow chamber section.

12. The treatment chamber according to claim 11, wherein each of the at least one hollow chamber section is an extruded section.

13. The treatment chamber according to claim 11, wherein each of the at least one hollow chamber section is an extruded aluminum section.

14. The treatment chamber according to claim 11, wherein each of the at least one hollow chamber section has an internal structure made of at least one of cross braces or sectional braces.

15. The treatment chamber according to claim 11, wherein the at least one hollow chamber section comprises a plurality of hollow chamber sections and wherein in each of the plurality of hollow chamber section at least one associated continuous cavity or passage is formed, wherein the at least one associated continuous cavity or passage of at least two of the plurality of hollow chamber sections fluid-communicate to form a line system for a temperature-controlling fluid, a treatment fluid or an air-conditioning fluid.

16. The treatment chamber according to claim 11, wherein the at least one hollow chamber section comprises two adjacent hollow chamber sections and wherein in each of the two adjacent hollow chamber section at least one associated continuous cavity or passage is formed, wherein the at least one associated continuous cavity or passage of the two adjacent hollow chamber sections fluid-communicate to form a line system for a temperature-controlling fluid, a treatment fluid or an air-conditioning fluid.

17. The treatment chamber according to claim 11, wherein each of the at least one hollow chamber section includes a connecting structure.

18. The treatment chamber according to claim 17, wherein the connecting structure is a groove-and-tongue type connection.

19. The treatment chamber according to claim 18, wherein the at least one hollow chamber section comprises two adjacent hollow chamber sections, and the adjacent hollow chamber sections are interconnected by the groove-and-tongue type connection in a fluid-tight manner.

20. The treatment chamber according to claim 19, wherein the connecting structure forms a sliding seat for compensating for tolerances between the two adjacent hollow chamber sections for weld mounting.

21. The treatment chamber according to claim 11, wherein the at least one hollow chamber section comprises a plural of interconnected hollow chamber sections forming a pre-fabricated component.

22. The treatment chamber according to claim 11, wherein the treatment chamber is a medical treatment chamber.

23. The treatment chamber according to claim 11, wherein the articles are medical articles, devices and equipment.

24. The treatment chamber according to claim 11, wherein the treatment chamber is used in a sterilization processes.
